(19) United States
(12) Patent Application Publication
(10) Pub. No.: US 2022/0319711 A1
(43) Pub. Date: Oct. 6, 2022

(54) Health Maintenance and Promotion Action Support Device, Health Maintenance and Promotion Action Support System, Recording Medium, and Health Maintenance and Promotion Action Support Method

(71) Applicant: ASUA Inc., Nagoya-shi, Aichi (JP)

(72) Inventors: Hiroshi Maji, Nagoya-shi, Aichi (JP); Takanari Miyamoto, Omaha, NE (US)

(21) Appl. No.: 17/616,020

(22) PCT Filed: Sep. 25, 2020
(86) PCT No.: PCT/JP2020/036281

§ 371 Date: Dec. 2, 2021

(30) Foreign Application Priority Data
Oct. 4, 2019 (JP) 2019-183979

(51) Int. Cl. G16H 50/30; G16H 10/20

(52) U.S. Cl. CPC G16H 50/30; G16H 10/20

(57) ABSTRACT

The health maintenance and promotion action support device is provided with a processor, the processor obtains action data pertaining to the user's health maintenance and promotion action including dental care; obtains evaluation data, pertaining to the health maintenance and promotion action, by the instructor instructing the user; and classifies the qualitative level of the user's health maintenance and promotion action based on the obtained action data and the evaluation data.

### CROSS-REFERENCE TO RELATED APPLICATIONS

This application is the national phase under 35 U.S.C. § 371 of PCT International Application No. PCT/JP2020/036281 which has an International filing date of Sep. 25, 2020 and designated the United States of America.

### FIELD

The present invention relates to a health maintenance and promotion action support device, a health maintenance and promotion action support system, a recording medium, and a health maintenance and promotion action assistance method.

### BACKGROUND

In recent years, the consciousness of health has been on the rise, and more and more people have been interested in early treatment of diseases, prevention of diseases, health care for health maintenance and promotion and the like. Although what is important in preventive care and health maintenance and promotion is people's consciousness in everyday life, there are many cases where health maintenance and promotion actions are lower in priority than others because of an external factor such as busyness and are neglected as a consequence. On the other hand, in the case of healthcare facilities, the current state is such that although it is possible to grasp the user's health condition when the user visits there and make a prediction therefrom, it is difficult to grasp the person's actual condition in daily life and no function to support fundamental prevention is possessed.

Japanese Laid-open Patent Publication No. 2002-132954 discloses a dental treatment support device capable of automatically creating an explanatory leaflet for explaining the treatment details to the patient by storing, in association with each other, medical practice groups where a plurality of associated medical practices are grouped by a series of dental treatment stages and comment information for explaining the treatment details to the patient.

### SUMMARY

The user's lifestyle habits are considered to be influenced by various factors such as the user's consciousness and efforts for health and way of thinking and mind when creating daily actions.

Therefore, even if the user's motivation for health maintenance and promotion actions is increased when the user receives an explanation or a guidance on the treatment details by using the device as in Japanese Laid-open Patent Publication No. 2002-132954, the motivation can be decreased under everyday environments by being influenced by an external factor that decreases the priority of health maintenance and promotion actions and this can decrease the quality of the health maintenance and promotion actions.

The present disclosure is made in view of such circumstances, and an object thereof is to provide a health maintenance and promotion action support device, a health maintenance and promotion action support system, a computer program and a health maintenance and promotion action support method capable of preventing the decrease in the motivation for health maintenance and promotion actions and improving the quality of the health maintenance and promotion actions.

A health maintenance and promotion action support device according to an embodiment of the present disclosure is provided with a processor, the processor obtains action data pertaining to a user's health maintenance and promotion action including dental care; obtains evaluation data, pertaining to the health maintenance and promotion action, by an instructor instructing the user; classifies a qualitative level of the user's health maintenance and promotion action based on the action data and the evaluation data.

A health maintenance and promotion action support system according to an embodiment of the present disclosure is provided with: the above-described health maintenance and promotion action support device; a user terminal that transmits the action data pertaining to the user's health maintenance and promotion action including dental care, to the health maintenance and promotion action support device; and an instructor terminal that transmits the evaluation data pertaining to the health maintenance and promotion action by the instructor instructing the user, to the health maintenance and promotion action support device.

A computer readable non-transitory recording medium recording a computer program according to an embodiment of the present disclosure causes a computer to execute: processing of obtaining action data pertaining to a user's health maintenance and promotion action including dental care; processing of obtaining evaluation data, pertaining to the health maintenance and promotion action, by an instructor instructing the user; and processing of classifying a qualitative level of the user's health maintenance and promotion action based on the obtained action data and evaluation data.

In a health maintenance and promotion action support method according to an embodiment of the present disclosure, action data pertaining to a user's health maintenance and promotion action including dental care is obtained; evaluation data, pertaining to the health maintenance and promotion action, by an instructor instructing the user is obtained; and a qualitative level of the user's health maintenance and promotion action is classified based on the obtained action data and evaluation data.

According to the present disclosure, it is possible to prevent the decrease in the user's motivation for the health maintenance and promotion actions and improve the quality of the health maintenance and promotion actions.

The above and further objects and features of the invention will more fully be apparent from the following detailed description with accompanying drawings.

### BRIEF DESCRIPTION OF THE DRAWINGS

FIG. 1 is a schematic view showing an example of the structure of a health maintenance and promotion action support system of the present embodiment.

FIG. 2 is a block diagram showing an example of the structure of a server.

FIG. 3 is a schematic view showing an example of a model of oral health condition changes.

FIG. 4 is a schematic view showing an example of the relationship among the gap, the compliance and the motivation.

FIG. 5 is an explanatory view showing examples of questions to the user.

FIG. 6 is an explanatory view showing examples of questions for evaluation by the instructor.

FIG. 7 is a schematic view showing an example of a result of a measurement of the periodontal pocket depth of maxillary teeth and mandibular teeth.

FIG. 8 is a schematic view showing an example of the input data and the output data of a classifier.

FIG. 9 is a schematic view showing a part of the structure of the classifier.

FIG. 10 is a schematic view showing an example of the support information DB.

FIG. 11 is a schematic view showing an example of the structure of a prediction portion.

FIG. 12 is a schematic view showing an example of the image of the oral health condition.

FIG. 13 is a schematic view showing an example of motivation changes.

FIG. 14 is a schematic view showing an example of the transition of the oral health condition when the health maintenance and promotion action support method of the present embodiment is used.

FIG. 15 is a schematic view showing an example of the temporal transition of the user's compliance.

FIG. 16 is a flowchart showing an example of the processing procedure of the health maintenance and promotion action support by the server.

FIG. 17 is a schematic view showing another example of the structure of the health maintenance and promotion action support device of the present embodiment.

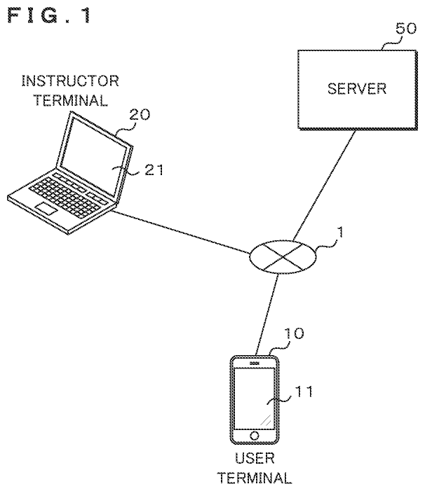

Hereinafter, an embodiment of the present disclosure will be described based on the drawings. FIG. 1 is a schematic view showing an example of the structure of a health maintenance and promotion action support system of the present embodiment. The health maintenance and promotion action support system is provided with: a server 50 as a health maintenance and promotion action support device; a user terminal 10 used by the user; and an instructor terminal 20 used by the instructor. While dental treatment, prevention and dental care will be described as an example of early treatment of diseases, prevention of diseases, health care for health maintenance and promotion and the like in the present description, the present invention is not limited to teeth. Moreover, in the present description, the qualitative level of the health maintenance and promotion actions is defined as “compliance.” While the user may be a patient visiting a dental office or a dental clinic, persons not visiting a dental office or a dental clinic but interested in dental care may be included. The user terminal 10, the instructor terminal 20 and the server 50 are interconnected through a communication network 1 such as the Internet. While only one user terminal 10 and one instructor terminal 20 are shown in FIG. 1 for convenience sake, more than one of each may be present. For the server 50, the function may be distributed by using a plurality of servers.

The user terminal 10 is provided with a display panel 11, and may be constituted, for example, by a smartphone, a tablet or a notebook personal computer. The user terminal 10 is capable of executing required processing with the server 50 by being installed with a health maintenance and promotion action support application.

The instructor terminal 20 is provided with a display panel 21, and is constituted, for example, by an information processing device such as a personal computer or a tablet. The instructor includes healthcare professionals such as dentists and dental hygienists. The instructor terminal 20 can be placed in dental offices and dental clinics. The instructor can input treatment details (the treatment outcome, the test result, etc.) pertaining to teeth by use of the instructor terminal 20.

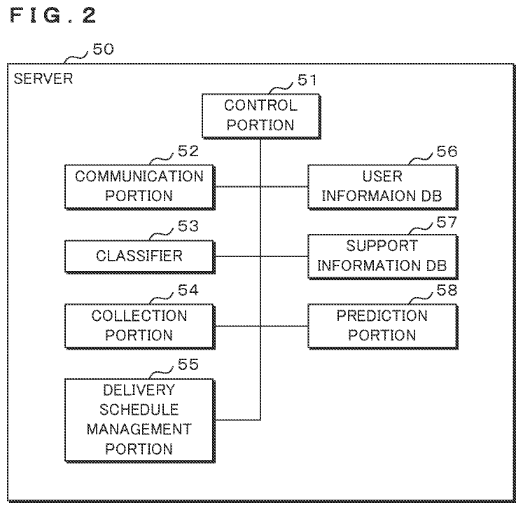

FIG. 2 is a block diagram showing an example of the structure of the server 50. The server 50 is provided with a control portion 51 that controls the entire part of the server 50, a communication portion 52, a classifier 53, a collection portion 54, a delivery schedule management portion 55, a user information DB 56, a support information DB 57 and a prediction portion 58. The control portion 51 may be constituted by a CPU, a ROM, a RAM and the like.

Before starting a detailed description of the server 50, first, regarding the prevention of the decrease in the user's motivation for the health maintenance and promotion actions and the improvement in the quality of the health maintenance and promotion actions by the server 50, the general outline of the contents of the inventors' idea will be described.

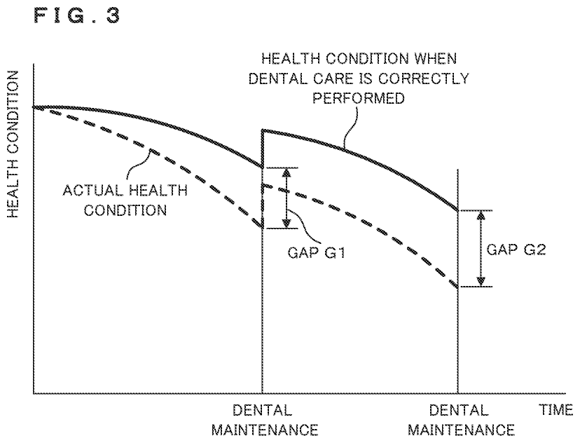

FIG. 3 is a schematic view showing an example of a model of oral health condition changes. In the figure, the vertical axis represents the health condition, and indicates that the health condition is better toward the upside. The horizontal axis represents the time. The graph shown by the solid line schematically shows changes in the health condition when dental care is correctly performed, and the graph shown by the broken line schematically shows changes in the actual health condition. The oral health condition is temporarily improved by a regular maintenance at a dental office (referred to also as dental maintenance). However, since sufficient care is impossible by the user's self-maintenance, the health condition tends to decline during the interval period when no dental maintenance is performed.

At the dental maintenance, it is assumed that there is a gap between the health condition when dental care is correctly performed and the actual health condition, by a medical examination and a checkup by a dentist or a dental hygienist. Regarding dental care, in the case of a user understanding his/her own constitution and environments and having correct knowledge about teeth, the above-mentioned gap can be considered small because it is considered that daily health maintenance and promotion actions (dental care) are appropriately performed. On the other hand, in the case of a user not understanding his/her constitution or environments and having little correct knowledge about teeth, the above-mentioned gap can be considered large because there are cases where appropriate care is not performed in actuality although the user thinks that he/she appropriately performs dental care and there are also cases where the user is indifferent to dental care. Moreover, the gap tends to increase as time proceeds (gap G1<gap G2).

That is, the qualitative level of the health maintenance and promotion actions such as the understanding of the own constitution and environments and correct knowledge about teeth can be defined, paradoxically, based on whether the gap is large or small. In the present description, the health maintenance and promotion actions are various actions associated with the user's health, includes various actions related to treatment and prevention of diseases and healthcare for health maintenance and promotion, and includes, for example, dental care. Under the condition where the compliance is high, it is considered that the health condition changes in a comparatively excellent state and the gap is small. Under the condition where the compliance is low, it is considered that more health condition deterioration than expected is brought about and the gap is large. If the compliance can be obtained, there is a possibility that the factor that creates the gap can be estimated and eliminated.

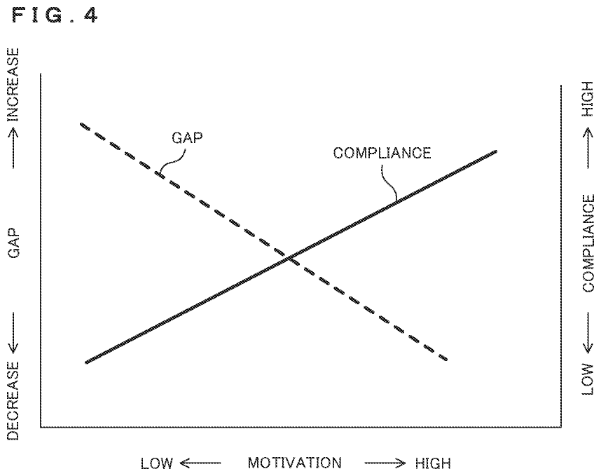

FIG. 4 is a schematic view showing an example of the relationship among the gap, the compliance and the motivation. While the compliance is the qualitative level (quality range) of the health maintenance and promotion actions and means a consciousness level in a comparatively long span, the motivation is the decision of the intention for daily health maintenance and promotion actions and means a short-term consciousness level. From the viewpoint of the user's daily psychological changes, the following is considered: If the motivation for the health maintenance and promotion actions decreases due to some external factor, the execution of appropriate health maintenance and promotion actions is inhibited and the priority of the health maintenance and promotion actions decreases, so that the quality of the health maintenance and promotion actions decreases to decrease the compliance. That is, as shown in FIG. 4, the compliance decreases as the motivation decreases. While the graphs are shown schematically by straight lines in the example of FIG. 4, they may be shown by curved lines. Moreover, since the gap and the compliance have a relationship such that one decreases as the other increases as mentioned above, the gap increases as the motivation decreases as shown in FIG. 4.

At the dental maintenance, it, is possible to continuously provide a support by a dentist or a dental hygienist to enable correct control by making each user (patient) understand his/her constitution and environments and eliminating the factor that decreases the motivation in daily life (external factor that decreases the priority of self-care) while providing the user with correct knowledge.

The health condition change model shown in FIG. 3 and the relationship among the gap, the compliance and the motivation shown in FIG. 4 apply not only to the field of dentistry but also to other fields. For example, in the health field of fitness, exercise for health corresponds to dental care and the oral condition corresponds to the body health condition and the physical condition. Moreover, it also corresponds to the sleeping condition, the supplement taking condition and the like.

The server 50 of the present embodiment simulatively reproduces an environment in which a dentist or a dental hygienist is always present in the interval period between a dental maintenance and the next dental maintenance, and eliminates the motivation decreasing factor in daily life to prevent the decrease in the motivation to improve the compliance. Hereinafter, details of the server 50 will be described.

The user information DB 56 is capable of recording information such as the user's ID, name, mail address, telephone number, action data, evaluation data, compliance index, delivered support information (an advice message and the delivery date) and health condition predicted value.

The communication portion 52 has the function as an action data obtaining portion and an evaluation data obtaining portion. The communication portion 52 provides the function of communication between the user terminal 10 and the instructor terminal 20 through the communication network 1.

The communication portion 52 is capable of obtaining, from the user terminal 10, the action data pertaining to the user's health maintenance and promotion actions including dental care. The action data is only necessarily data that enables the identification of the factor that influences the user's motivation for the health maintenance and promotion actions. More specifically, the server 50 is capable of transmitting questions as to the health maintenance and promotion actions to the user terminal 10 through the communication portion 52.

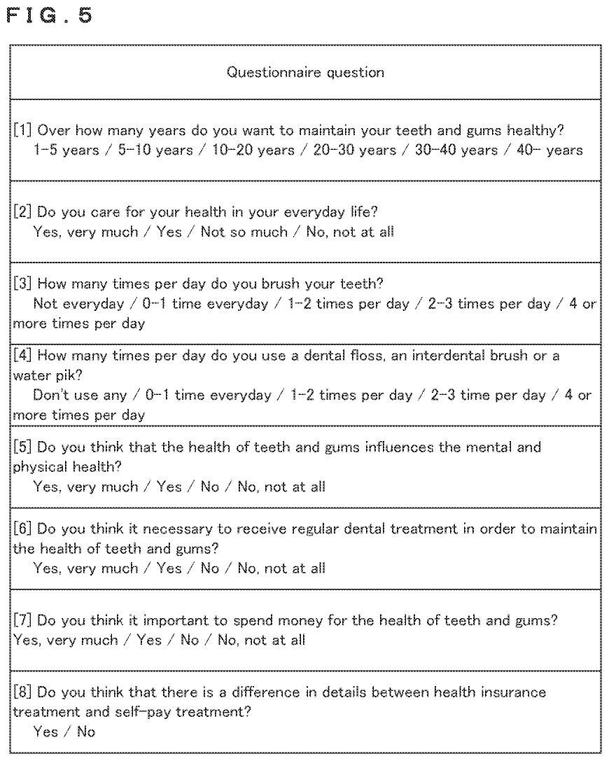

FIG. 5 is an explanatory view showing examples of questions to the user. The questions may be, for example, in the form of a questionnaire, and are only necessarily ones that enable the identification of the factor that influences the motivation for the health maintenance and promotion actions. For example, to a question “Over how many years do you want to maintain your teeth and gums healthy?” (question as to the consciousness of health), the user can provide an answer by selecting any of the choices “1-5 years”, “5-10 years”, “10-20 years”, “20-30 years”, “30-40 years” and “40 or more years”. Likewise, to a question “How many times per day do you brush your teeth?” (question as to the consciousness of actions), the user can provide an answer by selecting any of the choices “Not everyday”, “0-1 time everyday”, “1-2 times per day”, “2-3 times per day” and “4 or more times per day” Likewise, to a question “Do you think it important to spend money for the health of teeth and gums?” (question as to the consciousness of economy), the user can provide an answer by selecting any of the choices “Yes, very much.”, “Yes”, “No” and “No, not at all”. The same applies to the other questions. The questions and the choices of answers shown in FIG. 5 are examples and the present invention is not limited to the examples of FIG. 5.

The questions as shown in FIG. 5 are displayed on the display panel 11 of the user terminal 10, and the user can answer to the questions and send back the answers to the server 50. The questions may be transmitted, for example, when the instructor provides the first instruction and when a predetermined period has elapsed (for example, one month, three months, six months, one year) after the instruction. The communication portion 52 is capable of obtaining the answers to the questionnaire as the user's action data. This enables the identification of the factor that decreases the user's motivation at a desired time, whereby it is possible to carry out support measures to prevent the motivation from decreasing during a period when no instruction is provided by the instructor.

The communication portion 52 is capable of obtaining data pertaining to the evaluation of the user's health maintenance and promotion actions by the instructor, from the instructor terminal 20. As the evaluation data, for example, data in the medical records may be used, and subjective data by the instructor and medical data based on treatment and tests are included. The subjective data includes the user's answers to the questions by the instructor at the time of treatment and tests and the qualitative level of the user's health maintenance and promotion actions judged by the instructor (compliance as the instructor's impression). The medical data includes the depth of the periodontal pocket, tooth staining (plaque), BOP (Bleeding on Probing: bleeding at the time of probing).

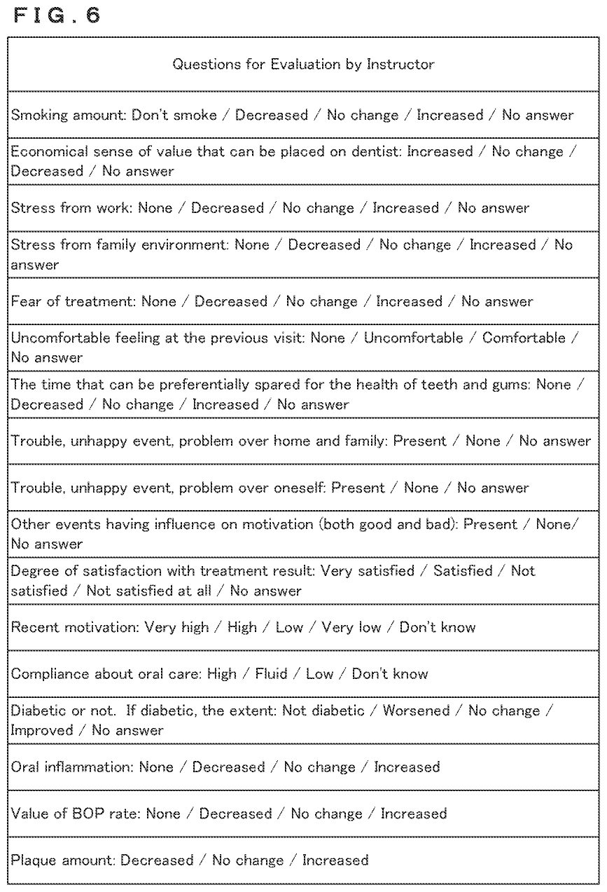

FIG. 6 is an explanatory view showing examples of questions for evaluation by the instructor. The questions shown in FIG. 6 are examples of the question items when a medical interview by the dentist or the dental hygienist is performed, and the answers of the user (patient) are recorded in the medical record. By obtaining answers from the user, the user's compliance as the instructor's impression can be estimated. The compliance as an impression can be expressed by a compliance index (for example, three classes of high, unstable and low, or ten classes of 1 to 10). The questions and the choices of answers shown in FIG. 6 are examples and the present invention is not limited to the examples of FIG. 6.

The questions and the choices of answers as shown in FIG. 6 are displayed on the display panel 21 of the instructor terminal 20, and by the instructor selecting answers, the answers are transmitted to the server 50 as subjective data.

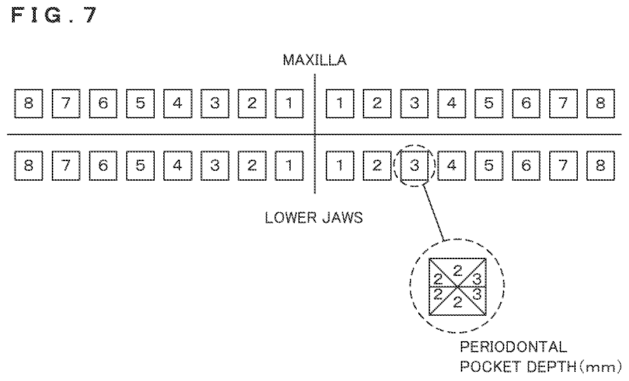

FIG. 7 is a schematic view showing an example of a result of a measurement of the periodontal pocket depth of maxillary teeth and mandibular teeth. The molar teeth, the premolar teeth, the canine teeth, the lateral incisors and the central incisors of the upper and lower jaws are expressed in a table form, and the periodontal pocket depths of six parts into which each tooth is divided are indicated by numerals. While the periodontal pocket depth of the left canine tooth of the lower jaw is shown in the example of FIG. 7 in the example of FIG. 7 for convenience sake, the periodontal pocket depths of the other teeth can be expressed in a similar way.

The teeth table form as shown in FIG. 7 is displayed on the display panel 21 of the instructor terminal 20, and by the instructor inputting or selecting the numeral of the periodontal pocket depth of each tooth, the numerals are transmitted to the server 50 as medical data. The medical data is not limited to the example of FIG. 7; the plaque of each tooth expressed in a table form can be displayed with a numeral or a color, or the presence or absence of BOP or the amount of bleeding of each tooth expressed in a table form can be displayed with a numeral or a color.

The classifier 53 classifies the compliance of the user's health maintenance and promotion actions (quantifies and classifies the compliance which is the qualitative level of the health maintenance and promotion actions) based on the action data and the evaluation data obtained through the communication portion 52. That is, the classifier 53 is capable of outputting the user's compliance index. While the classifier 53 may be constructed, for example, by a decision tree, different machine learning such as neural network may be used. In the present description, description will be given by using a decision tree as the classifier 53.

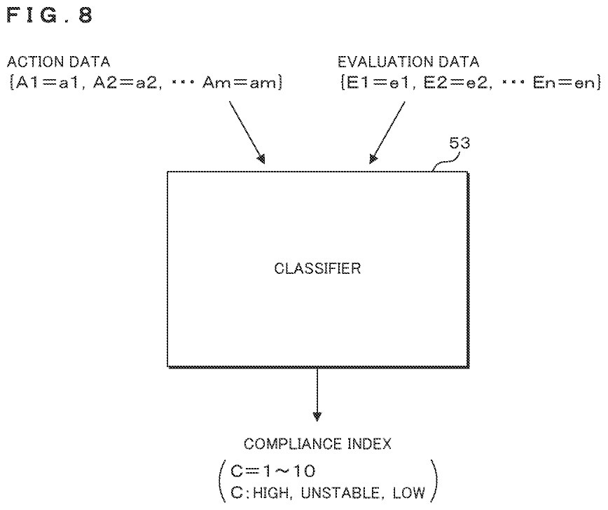

FIG. 8 is a schematic view showing an example of the input data and the output data of the classifier 53. The intention of the classifier 53 is to classify compliance (identify the compliance index) based on the input data expressed by pairs of attributes and their values. The input data can be divided into action data and evaluation data. The action data is constituted by a number, m, of pairs of an attribute A1 and its value a1, an attribute A2 and its value a2, , . . . , and an attribute Am and its value am. The attributes of the action data are, for example, the questions of the questionnaire shown in FIG. 5, and their values are the answers to the questionnaire. The values a1, a2, . . . , am may be character strings or may be ones converted into numerals.

The evaluation data is constituted by a number, n, of pairs of an attribute E1 and its value e1, an attribute E2 and its value e2, . . . , an attribute En and its value en. The attributes of the evaluation data are, for example, the questions shown in FIG. 6 and their values are the answers to the questions. Moreover, the attributes of the evaluation data are the compliance as the instructor's impression, and their values are the compliance indices. Moreover, the attributes of the evaluation data are the periodontal pocket depth, the plaque and BOP, and their values are measurement values thereof. The values e1, e2, . . . , en may be character strings or numerical values. Thereby, the qualitative level of the user's health maintenance and promotion actions can be classified based on medical and objective evaluation data.

By the action data and the evaluation data being inputted, the classifier 53 classifies the qualitative level of the user's health maintenance and promotion actions. As the classification of the qualitative level, for example, the compliance index C may be expressed by three classes (C: high, unstable, low) or the compliance index C may be expressed by ten classes (C=numerals 1 to 10).

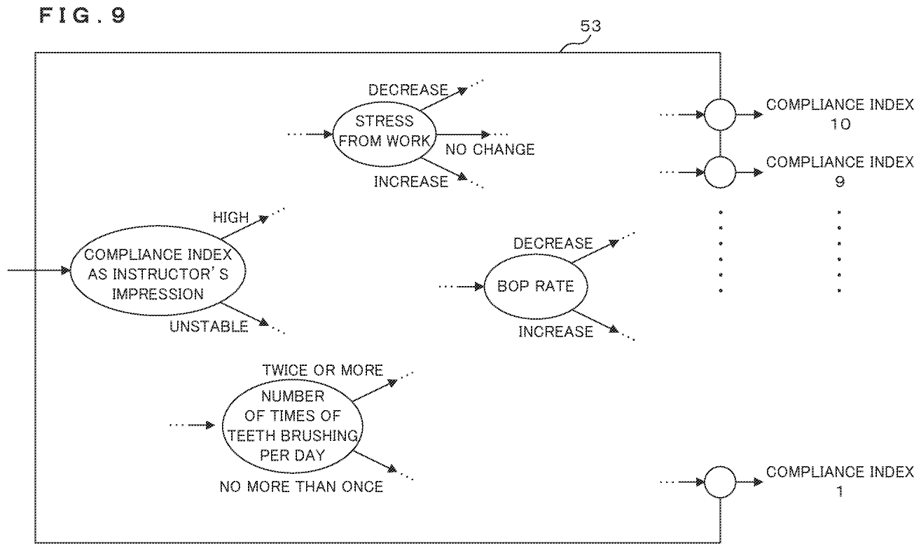

FIG. 9 is a schematic view showing a part of the structure of the classifier 53. The classifier 53 has a tree structure constituted by nodes and branches. To the nodes, the labels of the attributes are assigned, and to the branches arising from the nodes, values that the attributes can take are assigned. For example, to the node at the starting end, “compliance index as the instructor's impression” is assigned as the label of the attribute, and as the value that the attribute can take, two branches “high” and “unstable” are arising from the node, Moreover, as the label of the attribute of a node, “stress from work” is assigned, and as the values that the attribute can take, three branches of “decreased”, “no change” and “increased” are arising from the node. Moreover, as the label of the attribute of a node, “the number of times of teeth brushing per day” is assigned, and as the values that the attribute can take, two branches of “twice or more” and “not more than once” are arising from the nodes. Moreover, as the label of the attribute of a node, “the rate of BOP” is assigned, and as the value that the attribute can take, two branches of “decreased” and “increased” are arising from the node. Although not shown, the same applies to the other nodes and branches.

The use of the classifier 53 makes it possible to, starting with the attribute of the node at the starting end, follow branches one after another while testing the value of the attribute of each node and output the classification of the finally reached node at the end as the compliance index. The classifier 53 can be previously generated by use of teacher data. Specifically the tree structure of nodes and branches of the classifier 53 can be generated by providing the classifier with the labels of the attributes and the values of the attributes, and teacher data where a multiplicity of compliance index data at that time is collected. The teacher data includes the action data and the evaluation data, and the compliance index corresponding to the data.

By the above-described structure, rules of a mechanism linked with the compliance of the user's health maintenance and promotion actions can be established by associating the motivation decreasing factor that occurs in the user's daily health maintenance and promotion actions with the evaluation data, pertaining to the user's health maintenance and promotion actions, by the instructor. As a consequence, the compliance of the user's health maintenance and promotion actions can be classified by the medical and objective evaluation data in addition to the user's own subjective consciousness level. Thereby, the compliance of the user's health maintenance and promotion actions can be grasped; for example, the user's depth of understanding can be increased by optimizing the quality and amount of information to be provided according to the compliance index and adjusting the frequency. Moreover, by eliminating the motivation decreasing factor, the decrease in the motivation for the health maintenance and promotion actions is prevented, so that the quality of the health maintenance and promotion actions can be increased.

The delivery schedule management portion 55 which has the function as an output portion is capable of outputting support information pertaining to the user's health maintenance and promotion actions to the user terminal 10 according to the compliance index classified by the classifier 53. Next, the support information will be described.

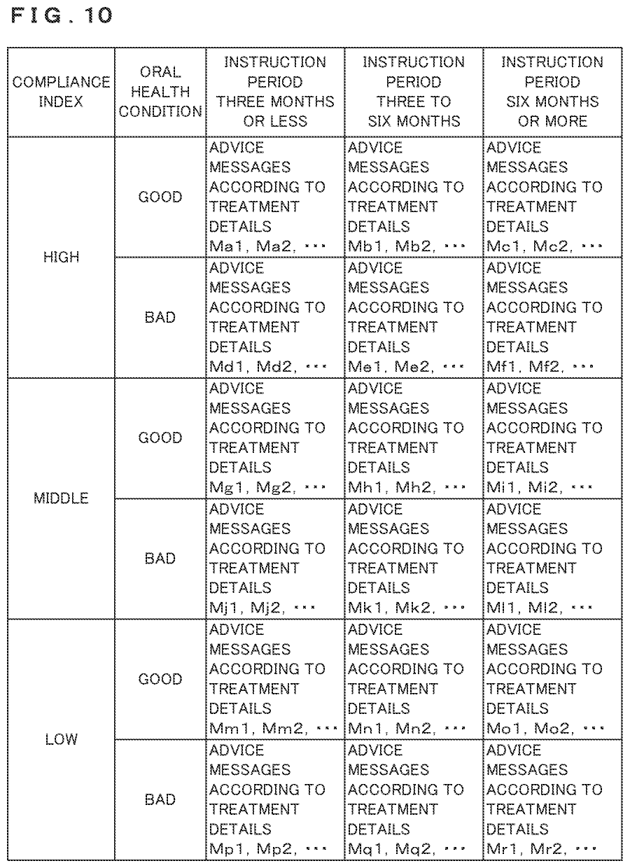

FIG. 10 is a schematic view showing an example of the support information DB 57. The support information includes advice messages. In the support information DB 57, advise messages are recorded that are associated with the compliance index, the oral health condition, the details of the treatment by the dentist or the dental hygienist and the instruction period. The support information DB 57 can be updated by the control portion 51.

As shown in FIG. 10, for example, when the compliance index outputted by the classifier 53 is “high” (for example, the compliance index C: high, or the compliance index C is 7 or higher), the oral health condition is “good” and the instruction period is three months or less, the delivery schedule management portion 55 can deliver any of the advice messages Ma1, Ma2, . . . to the user terminal 10 according to the treatment details.

When the compliance index outputted by the classifier 53 is “middle” (for example, the compliance index C: unstable, or the compliance index C is 4 to 6), the oral health condition is “good” and the instruction period is three months or less, the delivery schedule management portion 55 can deliver any of the advice messages Mg1, Mg2, . . . to the user terminal 10 according to the treatment details.

When the compliance index outputted by the classifier 53 is “low” (for example, the compliance index C: low, or the compliance index C is 3 or lower), the oral health condition is “good” and the instruction period is three months or less, the delivery schedule management portion 55 can deliver any of the advice messages Mm1, Mm2, . . . to the user terminal 10 according to the treatment details. Some of the advice messages may be the same advice message. The same applies to cases where the oral health condition is “bad” and where the instruction periods are three to six months and six months or more.

When the compliance index is comparatively high, an advice message advising to maintain the current health maintenance and promotion actions can be outputted. Further, respecting the user's own consciousness, high-level information such as the decrease in the frequency and the fulfillment of intellectual curiosity can be provided. When the compliance index is comparatively low, an advice message recommending actions that can eliminate the motivation decreasing factor can be outputted. By doing this, the user can receive support information in accordance with the compliance index of his/her own health maintenance and promotion actions, so that the decrease in the motivation can be prevented and the quality of the health maintenance and promotion actions can be increased. The support information can be outputted in the form of letters (text), charts, graphs, characters or avatars, or sound.

By installing an application for supporting the health maintenance and promotion actions on the user terminal 10, a chat function can be implemented between the server 50 and the user terminal 10. When the chat function is implemented, the server 50 can simulatively play the role of the dentist or the dental hygienist remotely. For example, the server 50 can transmit advice messages as shown in FIG. 10 to the user terminal 10. The advice messages may be accompanied by an image (for example, an oral image taken at the time of dental maintenance or an image obtained by processing said image) and a chart (for example, the result of the test at the time of dental maintenance) as needed. Moreover, a questionnaire may be delivered that imitates the diagnosis by the dentist or the dental hygienist for the purpose of periodically grasping the compliance. This questionnaire are also used for the purpose of discovering an external factor leading to a decrease in the motivation which factor occurs for each user. Viewing the advice message, the user can inputs the present situation of his/her health maintenance and promotion actions, the consciousness of teeth and the like in text format and return them to the server 50. Moreover, by answering the questionnaire, the user can receive periodical recalculation of the compliance and advice for eliminating the motivation decreasing factor in daily life. Such a chat between the user terminal 10 and the server 50 can be repetitively performed on a regular or irregular basis during the interval period between the last medical maintenance and the next medial maintenance. By performing language processing analysis of the text returned from the user terminal 10 and identifying with which of the attribute labels and their values of the classifier 53 the text is associated, the control portion 51 of the server 50 can make the text the input data to the classifier 53. Therefore, the classifier 53 can classify the compliance of the user's health maintenance and promotion actions even during the interval period between the last dental maintenance and the next dental maintenance. As described above, according to the present embodiment, quantification for objectively judging the qualitative level (compliance) pertaining to the user's health maintenance and promotion actions is realized and advice contents mainly including messages to prevent the decrease in the motivation in accordance therewith are provided, whereby the quality of the health maintenance and promotion actions can be increased. Moreover, for the dental hygienists who are in a position to be in charge of the patients' health maintenance and promotion actions at dental offices, the objective evaluation result of the qualitative level (compliance) of the health maintenance and promotion actions can be made good use of as one piece of information for the judgement for providing appropriate communication with the user and advice.

Next, a method of predicting the future of the oral health condition will be described.

The collection portion 54 collects the history data of the compliance index of the user's health maintenance and promotion actions classified by the classifier 53. The collection portion 54 is capable of collecting the classified compliance index as the history data in association with the date of classification every time the classifier 53 classifies the compliance index of the user's health maintenance and promotion actions.

The prediction portion 58 is capable of predicting the health condition including the condition of the user's teeth based on the collected history data. The prediction of the health condition may be the period (for example, the number of years) for which the teeth and the gums can stay healthy into the future or may be the health condition of the teeth and the gums at a point of time when a predetermined prediction period has elapsed (for example, after 10 years or 20 years from the present time). As the prediction portion 58, for example, a model may be used that is generated by machine learning such as deep learning.

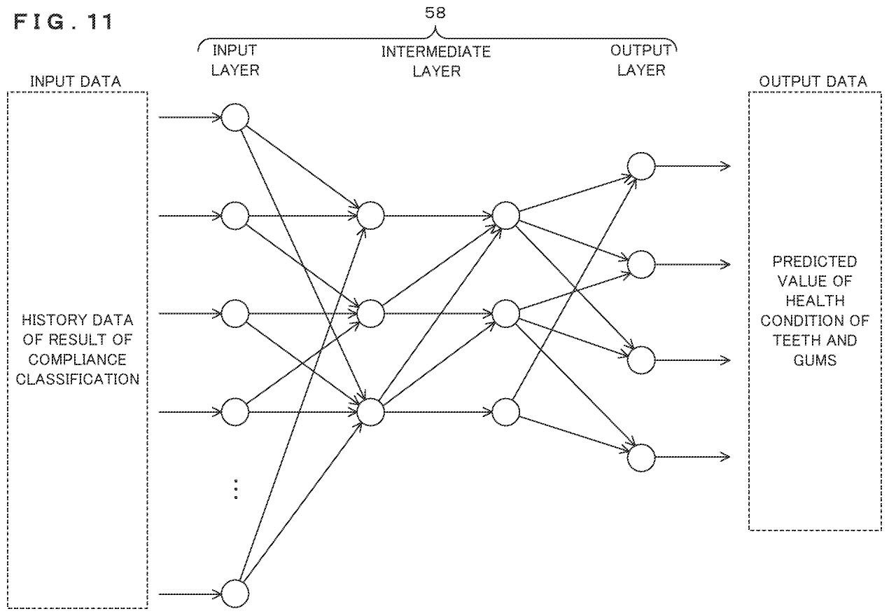

FIG. 11 is a schematic view showing an example of the structure of the prediction portion 58. The prediction portion 58 is a neural network model including deep learning, and while recurrent neural network model may be used as an example, a different model may be used. The prediction portion 58 is constituted by an input layer, an output layer and a plurality of intermediate layers. While two intermediate layers are shown in FIG. 11 for convenience sake, the number of intermediate layers is not limited to two and may be three or more.

In the input layer, the output layer and the intermediate layers, one or more than one node (neuron) is present, and the nodes in each layer is unidirectionally connected to the nodes being present in the preceding and succeeding layers with a desired weight. A vector having elements as many as the nodes in the input layer is provided as the input data of the prediction portion 58. The input data includes the history data of the result of the compliance classification. The history data may be time-series data. Moreover, although not shown, the input data may include the action data and the evaluation data. The output data includes predicted values of the health condition of the teeth and the gums. The predicted values may be expressed in text format, may be a quantified health condition, may express temporal changes of the numerals or may be an image (an illustration, a picture, etc.) showing the condition of the teeth and the gums.

For example, when the user's compliance makes transitions in the state of being low, the predicted value (expressed in text format) may be “Some of your teeth will be lost after five years from now.” When the user's compliance decreases with passing years, the predicted value (expressed in text format) may be “Some of your teeth will be lost after ten years from now.” When the user's compliance makes transitions in the state of being unstable, the predicted value (expressed in text format) may be “Your teeth can be lost after fifteen years from now.” and further, when the user's compliance makes transitions in the state of being high, the predicted value (expressed in text) may be “If you continue the current way, the health condition of your teeth will also be all right after ten years from now.”

Moreover, a character or an avatar expressing the user himself/herself may be preregistered so that when the health condition after 10 years or 20 years will become deteriorated, the facial expression of the character or the avatar is changed so as to express pain or distress and displayed on the user terminal 10 every delivery.

The prediction portion 58 can be structured by combining pieces of hardware such as a CPU (for example, a multiprocessor mounted with a plurality of processor cores), GPUs (Graphics Processing Units), DSPs (Digital Signal Processors) and FPGAs (Field-Programmable Gate Arrays). The prediction portion 58 is not limited to the neural network model but may be a different machine learning model.

For the learning by the prediction portion 58, history data of the compliance indices of a multiplicity of users' health maintenance and promotion actions and the health conditions corresponding to the history data may be used as the teacher data. For example, learning data may be used such that data pertaining to the temporal transition of a user's compliance index and the transition of the user's health condition are made a pair of learning data and such learning data is collected with respect to a multiplicity of users. Moreover, a multiplicity of users' action data and evaluation data, and the health conditions corresponding to the action data and the evaluation data may be used as the teacher data.

By doing this, the user's future health condition can be predicted, and support for maintaining or improving the quality of the health maintenance and promotion actions can be provided according to the predicted health condition. When delivering an advice message, the delivery schedule management portion 55 may deliver the predicted value of the health condition predicted by the prediction portion 58 together therewith.

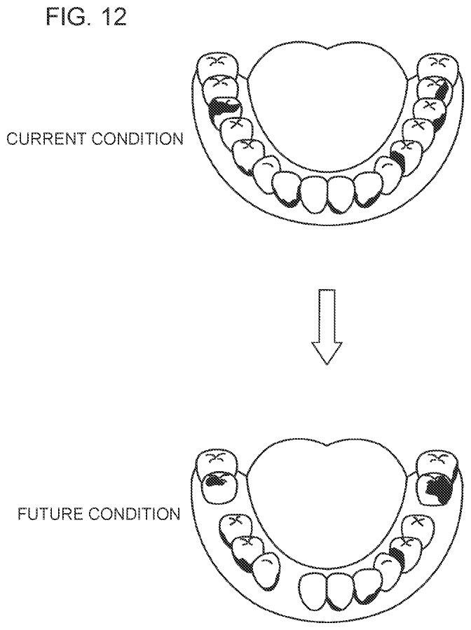

FIG. 12 is a schematic view showing an example of the image of the oral health condition. The current condition may be expressed by an actually taken image of the user's current oral cavity or may be schematically shown by a diagram. In the current condition, plaque, bad teeth and the like are observed at a plurality of positions. The future condition is, for example, a prediction of the oral condition after 10 years or 20 years from now and may be schematically expressed by a diagram or may be a processed actually taken image. In the future condition, bad teeth and the like become worse, and some teeth are lost. Shown the future condition of his/her teeth and gums with an actual image, the user can recognize the importance of correctly and regularly performing daily dental care. By doing this, the decrease in the user's motivation can be prevented to improve the compliance.

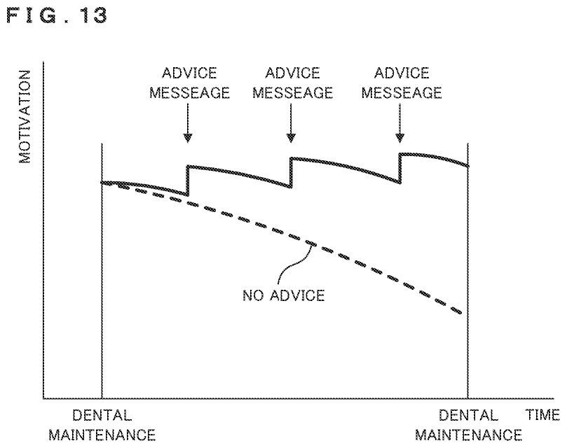

FIG. 13 is a schematic view showing an example of motivation changes. In the figure, the vertical axis represents the height of the motivation, and the horizontal axis represents time. The graph of the broken line indicates the motivation changes when there are no advice messages from the server 50, and the graph of the solid line indicates the motivation changes when there are advice messages from the server 50. When an external factor that leads to a decrease in the user's motivation occurs during the interval period between dental maintenances, the motivation decreases as indicated by the graph of the broken line, so that the priority of the user's health maintenance and promotion actions for teeth decreases. However, as shown by the graph of the solid line, by regularly and irregularly delivering to the user advice messages that can eliminate external factors that decrease the motivation, the user's motivation can be increased and the decrease in the user's motivation during the interval period can be suppressed.

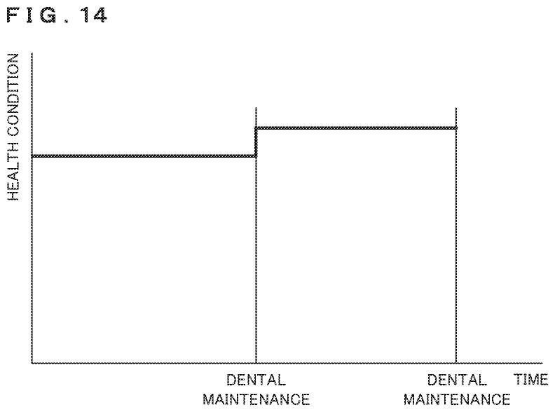

FIG. 14 is a schematic view showing an example of the transition of the oral health condition when the health maintenance and promotion action support method of the present embodiment is used. As shown in FIG. 13, since advice messages that can eliminate external factors that decrease the motivation are regularly or irregularly delivered to the user during the interval period between dental maintenances, the user's motivation is maintained comparatively high, and the user performs self-maintenance without any decrease in the priority of the health maintenance and promotion actions (dental care) for teeth and breaks a chain of health damages, so that the oral health condition is maintained. At the dental maintenance, professional treatment by the dentist or the dental hygienist is performed, so that the oral health condition is improved. Hybrid care management can be realized that is a combination of the dental care by the actual dentist or dental hygienist and the dental care by the simulative dentist or dental hygienist in cooperation with the server 50, which enables a low-cost and highly effective health maintenance and promotion action support method.

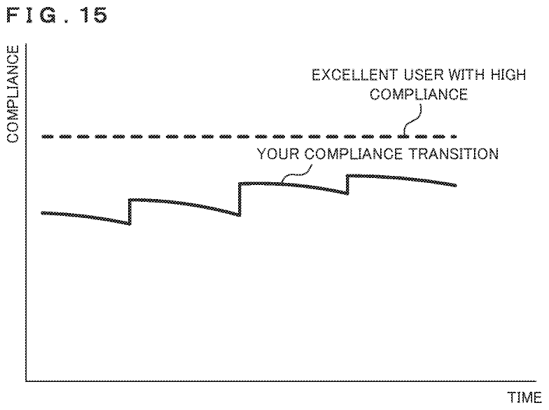

FIG. 15 is a schematic view showing an example of the temporal transition of the user's compliance. The temporal transition of the compliance shown in FIG. 15 can be displayed on the user terminal 10 as an example of the support information. While an example in which the compliance gradually increases with time is shown in the example of FIG. 15, the present invention is not limited thereto. Moreover, increase of the compliance in a staircase pattern may be made, for example, at the time of dental maintenance. By also displaying the compliance of an excellent user with high compliance as shown in FIG. 15, the user can easily grasp how much is the difference between the qualitative level of his/her own health maintenance and promotion actions and that of another user's, which can contribute to the improvement in the motivation for the health maintenance and promotion actions and the improvement in the oral health condition.

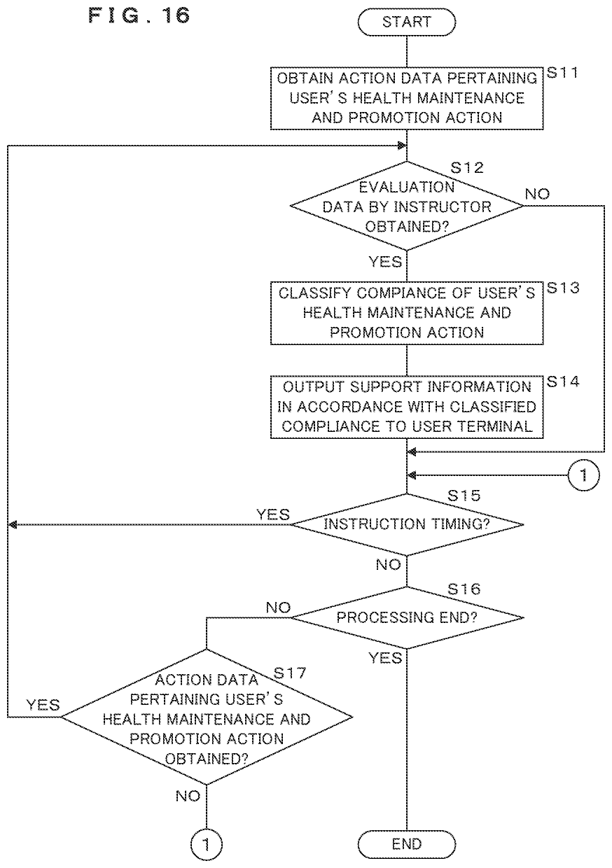

FIG. 16 is a flowchart showing an example of the processing procedure of the health maintenance and promotion action support by the server 50. In the following, for convenience sake, description will be given with the control portion 51 as the nucleus of the processing. The control portion 51 obtains action data pertaining to the user's health maintenance and promotion actions from the user terminal 10 (S11), and determines whether evaluation data by the instructor has been obtained or not (S12). When the evaluation data has not been obtained (NO at step S12), the control portion 51 performs the later-described processing of S15.

When the evaluation data by the instructor has been obtained (YES at S12), the control portion 51 classifies the compliance of the user's health maintenance and promotion actions (S13), and outputs (delivers) to the user terminal 10 the support information (advice message) in accordance with the classified compliance (S14). As the advice message, as shown in FIG. 10, not only the compliance index but also a desired one may be selected based on the oral health condition, the details of the treatment by the instructor, the delivery time and the like.

The control portion 51 determines whether it is the timing for instruction (for example, the timing for dental maintenance) or not (S15), and when it is the timing for instruction (YES at S15), the control portion 51 continues the processing of S12 and succeeding steps. When it is not the timing for instruction (NO at S15), the control portion 51 determines whether to end the processing or not (S16).

When the processing is not ended (NO at S16), the control portion 51 determines whether the action data pertaining to the user's health maintenance and promotion actions has been obtained or not (S17), and when the action data has been obtained (YES at S17), the control portion 51 continues the processing of step S12 and succeeding steps.

When the action data has not been obtained (NO at S17), the control portion 51 performs the processing of step S15. When the processing is ended (YES at S16), the control portion 51 ends the processing.

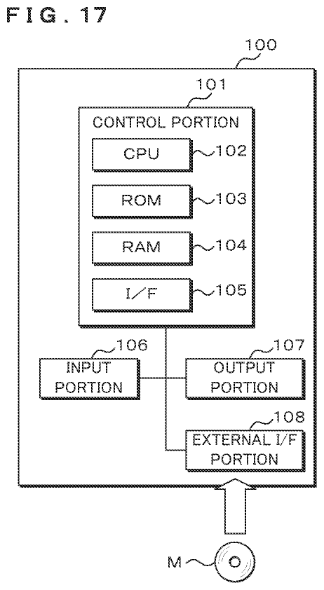

FIG. 17 is a schematic view showing another example of the structure of the health maintenance and promotion action support device of the present embodiment. In FIG. 17, reference numeral 100 represents a computer. The computer 100 is provided with a control portion 101, an input portion 106, an output portion 107, an external I/F (interface) portion 108 and the like. The control portion 101 is provided with a CPU 102, a ROM 103, a RAM 104, an I/F (interface) 105 and the like.

The input portion 106 is capable of obtaining the user's action data, the evaluation data by the instructor or information transmitted from the user terminal 10 and the instructor terminal 20. The output portion 107 is capable of outputting the support information. The I/F 105 has the function of interface between the control portion 101, and each of the input portion 106, the output portion 107 and the external I/F portion 108.

The external I/F portion 108 is a recording medium reading device such as a DVD reading device, and is capable of reading a computer program (for example, the processing procedure shown in FIG. 16) from a recording medium M (for example, a medium such as a DVD) recording the computer program. That is, the computer program is capable of causing the computer to execute the processing of obtaining the action data pertaining to the user's health maintenance and promotion actions including dental care, the processing of obtaining the evaluation data, pertaining to the health maintenance and promotion actions, by the instructor instructing the user, and the processing of classifying the qualitative level of the user's health maintenance and promotion actions based on the obtained action data and evaluation data.

Although not shown, the computer program recorded on the recording medium M is not limited to the one recorded on a portable medium but may include a computer program transmitted through the Internet or a different communication line.

The health maintenance and promotion action support device of the present embodiment is provided with: the action data obtaining portion that obtains the action data pertaining to the user's health maintenance and promotion action including dental care: the evaluation data obtaining portion that obtains the evaluation data, pertaining to the health maintenance and promotion action, by the instructor instructing the user; and the classifier that classifies the qualitative level of the user's health maintenance and promotion action based on the action data obtained by the action data obtaining portion and the evaluation data obtained by the evaluation data obtaining portion.

The health maintenance and promotion action support system of the present embodiment is provided with: the above-described health maintenance and promotion action support device; the user terminal that transmits the action data pertaining to the user's health maintenance and promotion action including dental care, to the health maintenance and promotion action support device; and the instructor terminal that transmits the evaluation data pertaining to the health maintenance and promotion action by the instructor instructing the user, to the health maintenance and promotion action support device.

The computer program of the present embodiment causes a computer to execute: the processing of obtaining the action data pertaining to the user's health maintenance and promotion action including dental care; the processing of obtaining the evaluation data, pertaining to the health maintenance and promotion action, by the instructor instructing the user; and the processing of classifying the qualitative level of the user's health maintenance and promotion action based on the obtained action data and evaluation data.

In the health maintenance and promotion action support method of the present embodiment, the action data pertaining to the user's health maintenance and promotion action including dental care is obtained, the evaluation data, pertaining to the health maintenance and promotion action, by the instructor instructing the user is obtained; and the qualitative level of the user's health maintenance and promotion action is classified based on the obtained action data and evaluation data.

The action data obtaining portion obtains the action data pertaining to the user's health maintenance and promotion actions including dental care. The health maintenance and promotion actions are various actions associated with the user's health, and includes various actions related to treatment and prevention of diseases and healthcare for health maintenance and promotion. The action data is only necessarily data that enables the identification of the factor that influences the user's motivation for the health maintenance and promotion actions. The motivation means the decision of the intention (short-term consciousness level) for daily health maintenance and promotion actions. In the case of dental care, the action data includes the health consciousness for teeth, the number of times of teeth brushing (action consciousness) and expenditure on teeth (economic consciousness).

The evaluation data obtaining portion obtains the evaluation data, pertaining to the user's health maintenance and promotion actions, by the instructor. In the case of dental care, the instructor includes dental hygienists and dentists. The evaluation data includes medical data based on treatment and tests and subjective data by the instructor. The medical data includes the depth of the periodontal pocket of the user's teeth and tooth staining (plaque). The subjective data includes the user's answers to the questions by the instructor at the time of treatment and tests and the qualitative level of the user's health maintenance and promotion actions judged by the instructor.

The classifier classifies the qualitative level of the user's health maintenance and promotion actions based on the obtained action data and evaluation data. While the classifier may be constructed, for example, by a decision tree, another machine learning such as neural network may be used. The qualitative level which is also referred to as compliance means the range of the quality of the daily health maintenance and promotion actions (medium-term consciousness level). The classification of the compliance can be expressed by a compliance index. The classifier can be previously generated by use of teacher data. The teacher data includes the action data and the evaluation data, and data of the qualitative level corresponding to the data. By the action data and the evaluation data being inputted, the classifier classifies the qualitative level of the user's health maintenance and promotion actions. As the classification of the qualitative level, for example, the compliance index may be expressed by three classes (high, unstable, low) or the compliance index may be expressed by ten classes (numerals 1 to 10).

By the above-described structure, rules of a mechanism linked with the qualitative level of the user's health maintenance and promotion actions can be established by associating the motivation decreasing factor that occurs in the user's daily health maintenance and promotion actions with the evaluation data, pertaining to the user's health maintenance and promotion actions, by the instructor. As a consequence, the qualitative level of the user's health maintenance and promotion actions can be classified by the medical and objective evaluation data in addition to the user's own subjective consciousness level. Thereby, the qualitative level of the user's health maintenance and promotion actions can be grasped; for example, since the motivation decreasing factor can be eliminated according to the qualitative level, the decrease in the motivation for the health maintenance and promotion actions is prevented, so that the quality of the health maintenance and promotion actions can be increased. The present embodiment may be applied to a system such as a treatment support system.

The health maintenance and promotion support device of the present embodiment is provided with the transmission portion that transmits a question as to the health maintenance and promotion actions to the user's user terminal, and the action data obtaining portion obtains the action data based on an answer to the question.

The transmission portion transmits questions as to the health maintenance and promotion actions to the user terminal. The questions may be, for example, in the form of a questionnaire, and are only necessarily ones that enable the identification of the factor that influences the motivation for the health maintenance and promotion actions. The questions may be transmitted, for example, when the instructor provides the first instruction and when a predetermined period has elapsed (for example, one month, three months, six months, one year) after the instruction. The action data obtaining portion can obtain the action data based on the answers to the questions. This enables the identification of the factor that decreases the user's motivation at a desired time, whereby it is possible to carry out support measures to prevent the motivation from decreasing during a period when no instruction is provided by the instructor.

In the health maintenance and promotion action support device of the present embodiment, the evaluation data obtaining portion obtains at least one of evaluation data pertaining to the user's teeth and evaluation data pertaining to the qualitative level of the user's health maintenance and promotion actions evaluated by the instructor.

The evaluation data obtaining portion obtains at least one of the evaluation data pertaining to the user's teeth and the evaluation data pertaining to the qualitative level of the user's health maintenance and promotion actions evaluated by the instructor. The evaluation data pertaining to teeth may be medical data such as the depth of the periodontal pocket of the user's teeth and tooth staining (plaque). The evaluation data pertaining to the qualitative level evaluated by the instructor includes the qualitative level of the user's health maintenance and promotion actions judged by the instructor. Thereby, the qualitative level of the user's health maintenance and promotion actions can be classified based on the medical and objective evaluation data.

The health maintenance and promotion action support device of the present embodiment is provided with the output portion that outputs support information pertaining to the user's health maintenance and promotion actions according to the qualitative level classified by the classifier.

The output portion outputs support information pertaining to the user's health maintenance and promotion actions according to the qualitative level classified by the classifier. The support information can be outputted to the user terminal. The support information may be outputted in the form of letters (text), charts, graphs, characters or avatars, or sound. When the qualitative level is comparatively high, support information advising to maintain the current health maintenance and promotion actions can be outputted. When the qualitative level is comparatively low, support information recommending actions that can eliminate the motivation decreasing factor can be outputted. By doing this, the user can receive support information in accordance with the qualitative level of his/her own health maintenance and promotion actions, so that the decrease in the motivation can be prevented and the quality of the health maintenance and promotion actions can be increased.

In the health maintenance and promotion action support device of the present embodiment, the output portion outputs support information including display information to display the temporal transition of the qualitative level classified by the classifier.

The output portion is capable of outputting support information including display information to display the temporal transition of the qualitative level classified by the classifier. Thereby, the user can grasp the transition of the qualitative level of his/her own health maintenance and promotion actions, and for example, deterioration of the oral health condition can be suppressed.

The health maintenance and promotion action support device of the present embodiment is provided with: the collection portion that collects the history data of the qualitative level of the user's health maintenance and promotion action, classified by the classifier: and the prediction portion that predicts the health condition including the condition of the user's teeth based on the history data collected by the collection portion.

The collection portion collects the history data of the qualitative level of the user's health maintenance and promotion action, classified by the classifier. The collection portion is capable of collecting the classified qualitative level as the history data in association with the date of classification every time the classifier classifies the qualitative level of the health maintenance and promotion actions.

The prediction portion predicts the health condition including the condition of the user's teeth based on the collected history data. The prediction of the health condition may be the period (for example, the number of years) for which the teeth and the gums can stay healthy into the future or may be the health condition of the teeth and the gums at a point of time when a predetermined prediction period has elapsed (for example, after 10 years or 20 years from the present time). As the prediction portion, for example, a model may be used that is generated by machine learning such as deep learning. For the learning by the prediction portion, history data of the qualitative levels of a multiplicity of users' health maintenance and promotion actions and the health conditions corresponding to the history data may be used as the teacher data. By doing this, the user's future health condition can be predicted, and support measures for maintaining or improving the quality of the health maintenance and promotion actions can be carried out according to the predicted health condition.

It is to be noted that, as used herein and in the appended claims, the singular forms “a”, “an”, and “the” include plural referents unless the context clearly dictates otherwise.

As this invention may be embodied in several forms without departing from the spirit of essential characteristics thereof, the present embodiments are therefore illustrative and not restrictive, since the scope of the invention is defined by the appended claims rather than by the description preceding them, and all changes that fall within metes and bounds of the claims, or equivalence of such metes and bounds thereof are therefore intended to be embraced by the claims.

1-9. (canceled)

10. A health maintenance and promotion action support device comprising:
a processor, the processor
obtains action data pertaining to a user's health maintenance and promotion action including dental care;
obtains evaluation data, pertaining to the health maintenance and promotion action, by an instructor instructing the user; and
classifies a qualitative level of the user's health maintenance and promotion action based on the action data and the evaluation data.

11. The health maintenance and promotion action support device according to claim 10, wherein the processor transmits a question as to the health maintenance and promotion action to the user's user terminal, and
obtains the action data based on an answer to the question.

12. The health maintenance and promotion action support device according to claim 10, wherein the processor obtains at least one of evaluation data pertaining to the user's teeth and evaluation data pertaining to the qualitative level of the user's health maintenance and promotion action evaluated by the instructor.

13. The health maintenance and promotion action support device according to claim 10, wherein the processor outputs support information pertaining to the user's health maintenance and promotion action according to the qualitative level.

14. The health maintenance and promotion action support device according to claim 13, wherein the processor outputs support information including display information to display a temporal transition of the qualitative level.

15. The health maintenance and promotion action support device according to claim 10, wherein the processor collects history data of the qualitative level of the user's health maintenance and promotion action; and
predicts a health condition including a condition of the user's teeth based on the history data.

16. A health maintenance and promotion action support system comprising: the health maintenance and promotion action support device according to claim 10; a user terminal that transmits the action data pertaining to the user's health maintenance and promotion action including dental care, to the health maintenance and promotion action support device; and an instructor terminal that transmits the evaluation data pertaining to the health maintenance and promotion action by the instructor instructing the user, to the health maintenance and promotion action support device.

17. A computer readable non-transitory recording medium recording a computer program causing a computer to execute:
processing of obtaining action data pertaining to a user's health maintenance and promotion action including dental care;
processing of obtaining evaluation data, pertaining to the health maintenance and promotion action, by an instructor instructing the user; and
processing of classifying a qualitative level of the user's health maintenance and promotion action based on the obtained action data and evaluation data.

18. A health maintenance and promotion action support method,
wherein action data pertaining to a user's health maintenance and promotion action including dental care is obtained;
evaluation data, pertaining to the health maintenance and promotion action, by an instructor instructing the user is obtained; and
a qualitative level of the user's health maintenance and promotion action is classified based on the obtained action data and evaluation data.
